(19) United States
(12) Patent Application Publication
(10) Pub. No.: US 2025/0210136 A1
(43) Pub. Date: Jun. 26, 2025

(54) Systems and Methods for Determining Validity of Indexes Attached to a Pool of Samples

(71) Applicant: ILLUMINA, INC., San Diego, CA (US)

(72) Inventors: Megan Blanchard, Poway, CA (US); Emily Parker, Encinitas, CA (US)

(21) Appl. No.: 18/979,073

(22) Filed: Dec. 12, 2024

Related U.S. Application Data
(60) Provisional application No. 63/613,317, filed on Dec. 21, 2023.

(51) Int. Cl. G16B 30/00; G16B 40/00

(52) U.S. Cl. CPC G16B 30/00; G16B 40/00

(57) ABSTRACT

Systems and methods are described for determining the validity of indexes attached to a pool of samples. A computing device receives genetic sequence data for each of a plurality of indices to be attached to a plurality of samples in a pool of samples. The computing device analyzes the genetic sequence data for each of the plurality of indices in the pool to determine whether the plurality of indices are compatible with each other. Then in response to determining that the plurality of indices are not compatible with each other, the computing device provides instructions to a user to replace at least one of the plurality of indices with a different index.

### CROSS-REFERENCE TO RELATED APPLICATIONS

This application claims priority to U.S. Provisional Patent Application No. 63/613,317, filed Dec. 21, 2023, entitled “Systems and Methods for Determining Validity of Indexes Attached to a Pool of Samples,” the entire disclosure of which is hereby expressly incorporated by reference herein.

### FIELD OF THE DISCLOSURE

The present disclosure generally relates to systems and methods for index validation.

### BACKGROUND

Conventionally, users are responsible for verifying that a pooling strategy, indices, and/or a sequencing devices are compatible for interpretable and reliable sequencing results. However, because of the range of products (e.g., library preparation kits, sequencing devices, indices) available to the user, sequencing runs with incompatibilities are common and result in inefficiencies and a waste of resources. For example, when there is an incompatibility in a sequencing run, the sequencing device may need to repeat the sequencing run which may be difficult when lab access time is limited. Repeating the sequencing run can also be costly and can cause delays which may be critical to patient outcomes in clinical labs. Moreover, some samples may be unique. In these instances, it can be very difficult if not impossible to obtain more of the sample if the original amount cannot be sequenced due to incompatibilities in the sequencing run.

For the foregoing reasons, there is a need for systems and methods for determining the validity of indexes attached to a pool of samples.

### BRIEF SUMMARY

The present embodiments relate to, inter alia, systems and methods for determining the validity of indexes attached to a pool of samples. Such systems and methods provide users with an effective and efficient solution for overcoming problems that arise when sequencing samples by determining compatibility and suggesting appropriate index substitution(s), if necessary, before a sequencing run. For example, the systems and methods described herein may be used to determine the compatibility of e.g., a user's pooling strategy, indices, and/or sequencing devices. Additionally, or alternatively, the systems and methods described herein may be used to determine the optimal sequencing run according to the user's specifications (e.g., available library preparation kits, indices, sequencing devices, etc.). Accordingly, the techniques described herein may enable a user to plan for interpretable and reliable sequencing results.

The systems and methods for determining the validity of indexes attached to a pool of samples may be implemented on one or more processors of a user computing device (e.g., such as one or more processors of a mobile device), one or more processors of a cloud-based computer or server, one or more processors of a sequencing device, and/or one or more processors of a library preparation system.

In one example aspect, a user application (app) may be downloaded or installed on a user computing device, such as a smart phone or tablet computer. A user may open the app to create a user profile. Creation of the profile may include a user providing or selecting preferences, such as allowing permissions for the app, via the computing device, to search local networks for devices such as a sequencing device and/or a library preparation system. In addition, creation of a profile may involve the user indicating preferences (e.g., preset(s)), such as sequencing device type(s), channel chemistry, inventory of library prep kits, etc.).

Accurate sequencing results rely on the appropriate use of indices for the specific sequencing run. To identify whether indices are compatible with each other and/or with the sequencing run, an index compatibility system may analyze characteristics of the sequencing run. These characteristics may include, for example, a number of libraries combined in a reaction and/or pool (also referred to herein as a “plexity” of the run), the sequencing device used in the run, the channel chemistry type used in the run, etc. For accurate results, indices must be chosen that are compatible with the sequencing run, and compatible with each other. For example, a sequencing run in which indices do not have a uniform length will not be accurate or reliable. In another example, a sequencing run that includes indices that differ by a single base pair or are identical may also be unreliable. In yet another example, a sequencing run having indices that lead to a color imbalance based on the planned sequencer type may be inaccurate or unreliable. The index compatibility system obtains the characteristics of the planned sequencing run and determines whether the chosen indices are compatible with the sequencing run and with each other. Additionally, the index compatibility system may analyze additional factors which influence the success of a sequencing run.

In various aspects, the user may provide the characteristics of a planned sequencing run to an index compatibility system (e.g., a cloud-based computer and/or server) by inputting the characteristics via a user interface (e.g., computing device). For example, the user may enter a library preparation kit type (which may include a list of indices, required reagents, consumables, etc.), a pooling strategy (which includes information regarding the samples in the pool and the arrangement of the pools in e.g., a 96 well plate), and a sequencing device type. Then, the index compatibility system may compare the characteristics of the planned sequencing run, check for compatibility of the characteristics, and, in some implementations, instruct the user to replace one or more indices so that all characteristics of the planned sequencing run are compatible. Additionally, or alternatively, the user may load consumables (which include the indices and necessary reagents for sequencing and/or attaching an index to a sample) into a library preparation system. The library preparation system may read an identification code (e.g., an RFID tag, a barcode, etc.) associated with each index to identify the indices. The index compatibility system may compare the index sequences for the planned sequencing run and check for compatibility of the indices while considering the sequencing device type and/or other characteristics of the sequencing run.

By assessing the compatibility of the indices planned to be attached to a pool of samples and replacing incompatible indices with a compatible set of indices, the index compatibility system increases the accuracy of the sequencing device. The index compatibility system also prevents from having to waste samples. Particularly when a sample is unique it can be very difficult if not impossible to obtain more of the sample if the original amount cannot be sequenced due to incompatibilities in the sequencing run. Additionally, the index compatibility system reduces the likelihood of having to repeat sequencing runs due to incompatibilities. This reduces costs and avoids delays which may be critical to patient outcomes in clinical labs. Furthermore, the index compatibility system advantageously prevents cross-contamination where reads from one sample get binned into another sample. This can be very dangerous and can lead to an incorrect diagnosis of a patient.

More specifically, as described herein, a method for determining the validity of indexes attached to a pool of samples is included. The method comprises receiving, at one or more processors, genetic sequence data for each index of a plurality of indices to be attached to a plurality of samples in a pool of samples. The method further comprises analyzing, by the one or more processors, the genetic sequence data for each of the plurality of indices in the pool to determine whether the plurality of indices are compatible with each other. The method further comprises, providing, by the one or more processors, instructions to a user to replace at least one of the plurality of indices with a different index in response to determining that the plurality of indices are not compatible with each other.

In addition, as described herein, a system for determining the validity of indexes attached to a pool of samples is included. The system comprises one or more processors and a non-transitory computer-readable memory storing instructions thereon. When executed by the one or more processors, the instructions cause the system to receive genetic sequence data for each of a plurality of indices to be attached to the plurality of samples in a pool of samples. The instructions further cause the one or more processors analyze the genetic sequence data for each of the plurality of indices in the pool to determine whether the plurality of indices are compatible with each other. In response to determining that the plurality of indices are not compatible with each other, the instructions cause the one or more processors to, provide, via the user interface, instructions to a user to replace at least one of the plurality of indices with a different index.

In addition, a tangible, non-transitory computer-readable memory storing instructions for determining the validity of indexes attached to a pool of samples is included. The instructions, when executed by one or more processors, cause the one or more processors to, for each of a plurality of indices to be attached to a plurality of samples in a pool of samples, receive genetic sequence data for the index. The instructions further cause the one or more processors to analyze the genetic sequence data for each of the plurality of indices in the pool to determine whether the plurality of indices are compatible with each other. In response to determining that the plurality of indices are not compatible with each other, the instructions cause the one or more processors to provide instructions to a user to replace at least one of the plurality of indices with a different index.

Advantages will become more apparent to those of ordinary skill in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### BRIEF DESCRIPTION OF THE DRAWINGS

The Figures described below depict various aspects of the system and methods disclosed therein. It should be understood that each Figure depicts an embodiment of a particular aspect of the disclosed system and methods, and that each of the Figures is intended to accord with a possible embodiment thereof. Further, wherever possible, the following description refers to the reference numerals included in the following Figures, in which features depicted in multiple Figures are designated with consistent reference numerals.

There are shown in the drawings arrangements which are presently discussed, it being understood, however, that the present embodiments are not limited to the precise arrangements and instrumentalities shown, wherein:

FIG. 1 illustrates an example computing system configured for determining the validity of indexes attached to a pool of samples, in accordance with various embodiments described herein.

FIG. 2 illustrates an example graphic user interface (GUI) which may be presented a display screen and/or user interface of a computing device, in accordance with various embodiments described herein.

FIG. 3 illustrates example index combinations for determining whether they would be compatible with each other, in accordance with various embodiments described herein.

FIG. 4 illustrates an isometric view of an example library preparation system, in accordance with various embodiments described herein.

FIG. 5 illustrates a flow diagram of an example method for determining the validity of indexes attached to a pool of samples, which can be implemented in a computing device, such as the user computing device of FIG. 1.

The Figures depict preferred embodiments for purposes of illustration only.

Alternative embodiments of the systems and methods illustrated herein may be employed without departing from the principles of the invention described herein.

### DETAILED DESCRIPTION

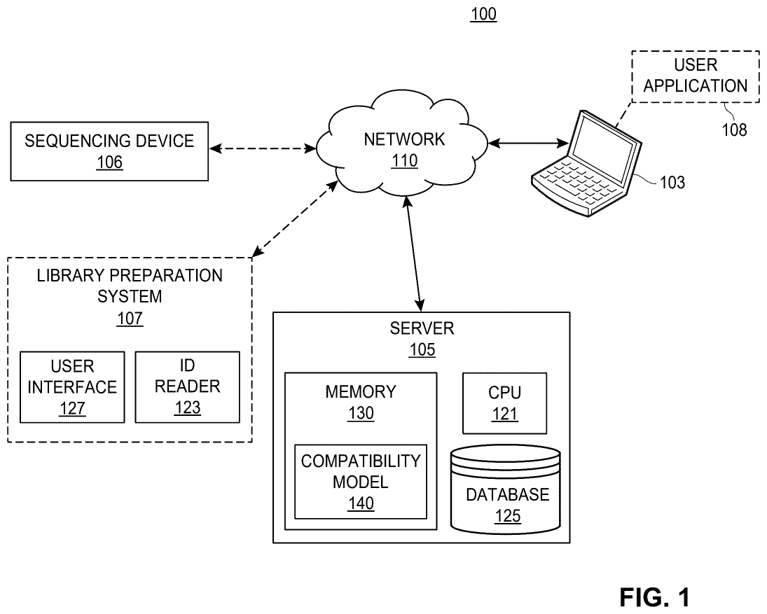

FIG. 1 illustrates a system 100 configured for determining the validity of indexes attached to a pool of samples, in accordance with various embodiments disclosed herein. In the illustrated example of FIG. 1, the system 100 includes a server 105, a user computing device 103, a network 110, a sequencing device 106, and a library preparation system 107. In various aspects, the server 105 may include multiple servers, which may include multiple, redundant, or replicated servers as part of a server farm. In still further aspects, the server(s) 105 may be implemented as cloud-based servers, such as a cloud-based computing platform. For example, the server(s) 105 may be any one or more cloud-based platform(s) such as Microsoft Azure®, Amazong AWS®, or the like. The server(s) 105 may include one or more processor(s) 121, one or more computer memories 130, and/or a database 125.

The memory 130 may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. The memory 130 may store an operating system (OS) (e.g., Microsoft Windows®, Linux, UNIX, etc.) capable of facilitating the functionalities, apps, methods, or other software as discussed herein. Additionally, or alternatively, genetic sequence data, sequencing run data, user data, etc., may also be stored in the memory 130 and/or in a database 125, which is accessible or otherwise communicatively coupled to the server(s) 105. In addition, the memory 130 may also store machine readable instructions, including any of one or more application(s), one or more software component(s), and/or one or more application programming interfaces (APIs), which may be implemented to facilitate or perform the features, functions, or other disclosure described herein, such as any methods, processes, elements or limitations, as illustrated, depicted, or described for the various flowcharts, illustrations, diagrams, figures, and/or other disclosure herein. It should be appreciated that one or more other applications may be executed by the processor(s) 121. It should be appreciated that given the state of advancements of computing devices, all of the processes functions and steps described herein with respect to the server 105 may be performed on a computing device (e.g., user computing device 103). In some implementations, some or all of the functionality of the server 105 can be implemented in the user computing device 103. In other implementations, some or all of the functionality of the user computing device 103 can be implemented in the server 105.

In some aspects, the memory 130 may store a compatibility model 140. The compatibility model 140 may include instructions that, when executed by the processor(s) s 121, may cause the processor(s) 121 to determine the compatibility of indices and/or compatibility of a plurality of sequencing run characteristics, as described herein. Additionally, or alternatively, the compatibility model 140 may include instructions, that when executed by the processor(s) s 121, may cause the processor(s) 121 to determine the optimal sequencing run for the samples and/or indices indicated by the user.

The user computing device 103 may be a portable device such as a smart phone or a tablet computer, for example. The user computing device 102 may also be a laptop computer, a desktop computer, a personal digital assistant (PDA), a wearable device such as a smart watch or smart glasses, a virtual reality headset, etc.

The user computing device 103 may include one or more processor(s) and a memory storing machine-readable instructions executable on the processor(s). The processor(s) may include one or more general-purpose processors (e.g., CPUs), and/or special-purpose processing units (e.g., graphical processing units (GPUs)). The memory can be, optionally, a non-transitory memory and can include one or several suitable memory modules, such as random access memory (RAM), read-only memory (ROM), flash memory, other types of persistent memory, etc.

The user computing device 103 may further include a network module, a user interface for receiving sequencing run data and displaying the results of a compatibility analysis, and an input/output (I/O) module. The network module may include one or more communication interfaces such as hardware, software, and/or firmware of an interface for enabling communications via a cellular network, a Wi-Fi network, or any other suitable network such as a network 110, discussed below. The I/O module may include I/O devices capable of receiving inputs from, and providing outputs to, the ambient environment and/or a user. The I/O module may include a touch screen, display, keyboard, mouse, buttons, keys, microphone, speaker, etc. In various implementations, the user computing device 103 can include fewer components or, conversely, additional components.

The user computing device 103 may communicate with the server 105, the library preparation system 107, and/or the sequencing device 106 via a network 110. The network 110 may include one or more of an Ethernet-based network, a private network, a cellular network, a local area network (LAN), and/or a wide area network (WAN), such as the Internet. In certain aspects, the network 110 may include any communication link suitable for short-range communications and may conform to a communication protocol such as, for example, Bluetooth™ (e.g., BLE), Wi-Fi (e.g., Wi-Fi Direct), etc. Additionally, or alternatively, the network 110 may be, for example, Wi-Fi, a cellular communication link (e.g., conforming to 3G, 4G, or 5G standards), etc. In some scenarios, the network 110 may also include a wired connection.

The memory may store instructions for implementing a user application 108 that can receive sequencing data for a sequencing run from a user. For example, the user may enter the library preparation kit type (which includes a list of indices, required reagents, consumables, etc.), the pooling strategy (which includes information regarding the samples in the pool and the arrangement of the pools in for example a 96 well plate), and the sequencing device type via the user interface. In other implementations, the user may load consumables (which include the indices and necessary reagents for sequencing and/or attaching an index to a sample) into the library preparation system 107. The library preparation system 107 may read an identification code (e.g., an RFID tag, a machine-readable barcode, etc.) associated with each index to identify the indices. Then the library preparation system 107 may transmit the sequencing data for the sequencing run to the user application 108 and/or the server device 105.

Then the user application 108 may transmit characteristics of the planned sequencing run to the server 105 to check for compatibility of the characteristics. The user application 108 may then receive the results of the compatibility analysis from the server 105. In other implementations, the user application 108 may perform the compatibility analysis based on the characteristics of the planned sequencing run.

Then the user application 108 may present the results of the compatibility analysis on the user interface. For example, the results may be that the indices are compatible and the user application 108 may present instructions to move forward with the sequencing run. In another example, the results may be that the indices are incompatible and the user application 108 may present instructions to replace at least one of the plurality of indices with a different index. The user application 108 may also present a recommendation on how to resolve the incompatibility. For example, the recommendation may be to replace index “N712” with index “D705.” In some implementations, the user application 108 may transmit instructions to the library preparation system 107 via the network 110 to cause the library preparation system 107 to attach the different index to one of the samples in the pool. Then the library preparation system 107 may attach the different index to one of the samples in the pool. In other implementations, the user application 108 is implemented in a computing device within the library preparation system 107, such as the controller, as described in more detail below. Then the controller transmits control signals to components within the library preparation system 107 to attach the different index to one of the samples in the pool, thus ensuring that the indices used in the sequencing run are the compatible indices.

The library preparation system 107 can be used to automatically, easily, and efficiently prepare DNA libraries for sequencing applications, for example. The library preparation system 107 may be used in conjunction with the server 105 and/or user computing device 103 to obtain sequencing run data to analyze the indices in a sequencing run for compatibility, and to attach at least one different index to a sample in the sequencing run if the indices originally included in the planned sequencing run are deemed incompatible.

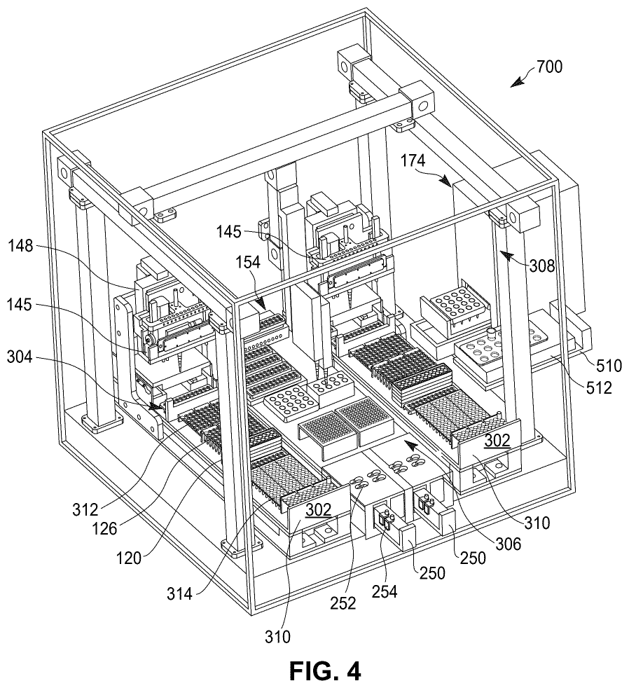

The library preparation system 107 includes a consumables area, a first working area, a second working area, and a loading area. The second working area also includes a consumables area. The consumables area and the first working area may be referred to as a first bay (e.g., a first assay bay) and the second working area may be referred to as a common bay. The library preparation system 107 may include any number of consumables areas and a corresponding number of first working areas. The library preparation system 107 may include four consumables areas and four first working areas or the library preparation system 107 may include two consumables areas and two first working areas as shown in FIG. 4. Other numbers of working areas may be used. If more than one consumables area/first working area are included, the library preparation system 107 can perform a corresponding number of workflows at the same time and/or at different times. One workflow (e.g., one assay) may be performed at one of the first working areas and another workflow (e.g., a second assay) can be performed at another one of the first working areas as an example.

FIG. 4 illustrates an isometric view 700 of the library preparation system 107. The library preparation system 107 may perform DNA library preparation workflows that include amplification processes, cleanup processes, quantification processes, library normalization processes, pooling processes, denaturing processes, and/or diluting processes in some implementations. The loading area 308 may be associated with loading and/or transferring a prepared sample to a system such as a sequencing system and/or a next generation sequencing system. The first working area 304 may be associated with amplification processes and cleanup processes and the second working area 306 may be associated with quantification processes, library normalization processes, pooling processes, denaturing processes, and/or diluting processes.

The library preparation system 107 may perform different workflows. The workflows may include whole genome sequencing (WGS) workflows, DNA & RNA enrichment workflows, methylation workflows, split-pool amplicon workflows, amplicon workflows, exome sequencing workflows, ChIP-seq workflows, Methyl-Seq workflows, metagenomic, mate-pair workflows, single-cell workflows, cDNA workflows, ligation workflows, adapter ligation workflows, tagmentation workflows, multiplexing workflows, and/or long-read workflows, as examples. The DNA library preparation workflow can be performed on any number of samples such as between one sample and twenty four samples. The library preparation system 107 thus allows for variable batch processing.

The consumable area(s) 302 may be used to load and store reagents and consumables 120, 126 needed for a library preparation process, including, disposable tips, wet or dry assay specific reagents, wet or dry bulk reagents, and reaction plates and wells. The consumables area 302 includes a consumables receptacle 310 that receives a tip tray having a first tip and a second tip, a first plate having a well containing a sample, and a second plate having a well. The consumables receptacle 310 may be a drawer that can be pulled out from the library preparation system 107 and loaded with the consumables 120, 126. The consumables receptacle 310 also includes a lid, an index tray having a well containing indexes, a bead tray having a well containing beads, a liquid reservoir 312, and a dry reagent reservoir 314. One or more of these reagents may be lyophilized and included with the dry reagent reservoir 314. The second working area 306 may also have a tip tray and a third plate having a well.

The library preparation system 107 includes a mover and the first working area 304 includes a contact dispenser 145, a stage 148, a magnet, and a thermocycler. The contact dispenser 145 may be movable to aspirate/dispense liquid to the consumables area 302 and/or to the first working area 304.

The stage 148 may be an x-z stage, such that the stage 148 is movable in the x and z directions (but not in the y direction). The stage 148 and the contact dispenser 145 may be movable to aspirate and/or dispense fluid between and above the consumables area 302 and the first working area 304 as a result. The contact dispenser 145 may, for example, move linearly in the x direction, which thereby reduces the risk of cross-contamination (between different samples) and allows some or all of the tips employed in the library preparation system 107 to be reusable for at least part of the processes performed by the library preparation system 107.

The second working area 306 includes an analyzer area 154, and the library preparation system 107 also includes a contact dispenser 318 and a stage. The stage may be referred to as a cross-bay gantry. The contact dispenser 318 may additionally or alternatively be implemented by a non-contact dispenser for aspirating/dispensing throughout the library preparation system 107. The dispenser and the stage can operate in the first working area 304 and the second working area 306.

The contact dispenser 145 may be movable to aspirate/dispense liquid to the consumables area 302, to the first working area 304, and/or to the second working area 306. The contact dispenser 145 may carry two tips (or two sets of tips) in some implementations, where one of the tips can hold a first volume of fluid and the other one of the tips can hold a second volume of fluid. The contact dispenser 145 may include two contact dispensers, where each dispenser carries one of tips. The two contact dispensers may be independently movable relative to one another. The first volume may be about 50 microliters and the second volume may be about 500 microliters.

The stage may be an x-y-z stage, such that the mover is movable in the x, y, and z directions. The stage and the contact dispenser 145 may be movable to aspirate and/or dispense fluid between and above the consumables area 302, the first working area 304, and/or the second working area 306 as a result.

The mover may include a robotic arm and/or include grippers. The stage may carry the mover and the contact dispenser 145 in some implementations.

The loading area 308 includes a sipper assembly 174. The sipper assembly 174 may be referred to as a sample sipper manifold assembly or a sample sipper assembly. The sipper assembly 174 may include sippers. Any number of sippers may be included such as between two sippers and sixteen sippers as an example. The sipper assembly 174 may be coupled to a corresponding number of the flow cells of the sequencing device 106 via sippers. The sipper assembly 174 includes a plurality of ports in some implementations where each port of the sipper assembly 174 may receive one of the sippers. The sippers may be referred to as fluidic lines. The sipper assembly 174 also includes a valve that may be selectively actuated to control the flow of fluid through a fluidic line. The fluidic line may be referred to as a sample sipper assembly. The sipper assembly 174 also includes a pump to selectively flow the prepared sample from a well through the sipper, through the fluidic line, and out of the library preparation system 107 to the sequencing device 106. The sequencing device 106 may be used to perform an analysis on one or more samples of interest. The sample may include one or more DNA clusters that are linearized to form a single stranded DNA (sstDNA).

In one example workflow, the stage aligns the contact dispenser 145 with the index tray and the contact 145 dispenser aspirates the indexes from the index tray using the first tip. The stage can then align the contact dispenser 145 with the first plate and the contact dispenser 145 dispenses the indexes into the well of the first plate. The mover moves the lid from the consumables area 302 and places the lid on the first plate to cover the well of the first plate with the lid. In this manner, the library preparation system 107 attaches selected indices to the pool of samples within the well (e.g., indices which are identified as compatible with each other).

The library preparation system 107 may perform pooling processes to pool the samples with attached indices. The stage aligns the contact dispenser 145 with the tip tray of the second working area 306 and the contact dispenser 145 places the tip in the tip tray and the contact dispenser 145 then couples with another tip from the tip tray to initiate the pooling processes in some implementations. The mover moves a plate from the second working area 306 to the plate receptacle of the second working area 306. The stage aligns the contact dispenser 145 with the second plate and the contact dispenser 145 aspirates the sample from the well of the second plate. The stage then aligns the contact dispenser 145 with the third plate and the contact dispenser 145 dispenses the sample into the well of the third plate. Additional samples from other wells of the second plate may be deposited into the well of the third plate in a similar manner to combine a plurality of samples together. A single tip can be used for the pooling processes. The contact dispenser 145 may pipette from final archive library well directly to pool and, thus, unique tips per sample may be used.

The library preparation system 107 also includes a controller including a user interface, a communication interface, one or more processors, and a memory storing instructions executable by the one or more processors to perform the various functionality discussed herein. The user interface, the communication interface, and the memory are electrically and/or communicatively coupled to the one or more processors. In some implementations, the controller is located in the same area as the other components of the library preparation system 107 and may be physically coupled to the other components of the library preparation system 107, for example via a wired connection. In other implementations, the controller is located remotely from the other components of the library preparation system 107 and may be communicatively coupled to the other components of the library preparation system 107, for example via a wireless connection. For example, the controller may be implemented on a cloud computing device. Some or all of the functionality of the user computing device 103 or the server 105 may be implemented on the controller.

In an implementation, the user interface receives input from a user and provides information to the user associated with the operation of the library preparation system 107 (e.g., information about the analysis taking place). The user interface library preparation system 107 may include a touch screen, a display, a key board, a speaker(s), a mouse, a track ball, and/or a voice recognition system. The touch screen and/or the display may display a graphical user interface (GUI).

In an implementation, the communication interface enables communication between the library preparation system 107 and remote systems (e.g., the server 105, the user computing device 103, the sequencing device 106, etc.) using the network 110.

The one or more processors may include one or more of a processor-based system(s) or a microprocessor-based system(s). In some implementations, the one or more processors includes a reduced-instruction set computer(s) (RISC), an application specific integrated circuit(s) (ASICs), a field programmable gate array(s) (FPGAs), a field programmable logic device(s) (FPLD(s)), a logic circuit(s), and/or another logic-based device executing various functions including the ones described herein.

The memory can include one or more of a hard disk drive, a flash memory, a read-only memory (ROM), erasable programmable read-only memory (EPROM), electrically erasable programmable read-only memory (EEPROM), a random-access memory (RAM), non-volatile RAM (NVRAM) memory, a compact disk (CD), a digital versatile disk (DVD), a cache, and/or any other storage device or storage disk in which information is stored for any duration (e.g., permanently, temporarily, for extended periods of time, for buffering, for caching).

The sequencing device 106 may be a sequencing system and/or a next generation sequencing (NGS) system. The sequencing device 106 can be used to perform an analysis on one or more samples of interest. The sample may include one or more DNA clusters that have been linearized to form a single stranded DNA (sstDNA). In some implementations, the sequencing device 106 is adapted to receive a pair of flow cell assemblies including corresponding flow cells and includes, in part, an imaging system and a flow cell interface having flow cell receptacles that support the corresponding flow cell assemblies. The flow cell interface may be associated with and/or referred to as a flow cell deck structure. The sequencing device 106 also includes a stage assembly, a pair or reagent selector valve assemblies that each include a reagent selector valve and a valve drive assembly, and a controller. The reagent selector valve assemblies may be referred to as mini-valve assemblies. The controller is electrically and/or communicatively coupled to the imaging system, reagent selector valve assemblies, and to the stage assembly and is adapted to cause the imaging system, reagent selector valve assemblies and the stage assembly to perform various functions.

Referring to the flow cells, each of the flow cells includes a plurality of channels, each having a first channel opening positioned at a first end of the flow cell and a second channel opening positioned at a second end of the flow cell. Depending on the direction of flow through the channels, either of the channel openings may act as an inlet or an outlet. The flow cells may include any number of channels (e.g., 1, 2, 6, 8).

In some such implementations, one or more of the nucleotides has a unique fluorescent label that emits a color when excited. The color (or absence thereof) is used to detect the corresponding nucleotide. In the implementation shown, the imaging system excites one or more of the identifiable labels (e.g., a fluorescent label) and thereafter obtains image data for the identifiable labels. The labels may be excited by incident light and/or a laser and the image data may include one or more colors emitted by the respective labels in response to the excitation. The image data (e.g., detection data) may be analyzed by the sequencing device 106. The imaging system may be a fluorescence spectrophotometer including an objective lens and/or a solid-state imaging device. The solid-state imaging device may include a charge coupled device (CCD) and/or a complementary metal oxide semiconductor (CMOS). However, other types of imaging systems and/or optical instruments may be used. For example, the imaging system may be or be associated with a scanning electron microscope, a transmission electron microscope, an imaging flow cytometer, high-resolution optical microscopy, confocal microscopy, epifluorescence microscopy, two photon microscopy, differential interference contrast microscopy, etc.

Referring to the controller, the controller includes a user interface, a communication interface, one or more processors, and a memory storing instructions executable by the one or more processors to perform various functions including the disclosed implementations. The user interface, the communication interface, and the memory are electrically and/or communicatively coupled to the one or more processors.

In an implementation, the communication interface 992 is adapted to enable communication between the sequencing device 106 and remote systems (e.g., the user computing device 103, the server 105, the library preparation system 107, etc.) via the network 110.

The one or more processors may include one or more of a processor-based system(s) or a microprocessor-based system(s). In some implementations, the one or more processors includes one or more of a programmable processor, a programmable controller, a microprocessor, a microcontroller, a graphics processing unit (GPU), a digital signal processor (DSP), a reduced-instruction set computer (RISC), an application specific integrated circuit (ASIC), a field programmable gate array (FPGA), a field programmable logic device (FPLD), a logic circuit, and/or another logic-based device executing various functions including the ones described herein.

The memory can include one or more of a semiconductor memory, a magnetically readable memory, an optical memory, a hard disk drive (HDD), an optical storage drive, a solid-state storage device, a solid-state drive (SSD), a flash memory, a read-only memory (ROM), erasable programmable read-only memory (EPROM), electrically erasable programmable read-only memory (EEPROM), a random-access memory (RAM), a non-volatile RAM (NVRAM) memory, a compact disc (CD), a compact disc read-only memory (CD-ROM), a digital versatile disk (DVD), a Blu-ray disk, a redundant array of independent disks (RAID) system, a cache and/or any other storage device or storage disk in which information is stored for any duration (e.g., permanently, temporarily, for extended periods of time, for buffering, for caching).

In some implementations, the user computing device 103 or the server 105 may communicate with the sequencing device 106 via the network 110 to receive sequencing device type information from the sequencing device 106. Then the user computing device 103 or the server 105 may use the sequencing device type information when analyzing the indices for compatibility. The sequencing device 106 may also provide sequencer data, sequencing run data, read data, etc., to the user computing device 103 or the server 105, which may be used to analyze the indices for compatibility.

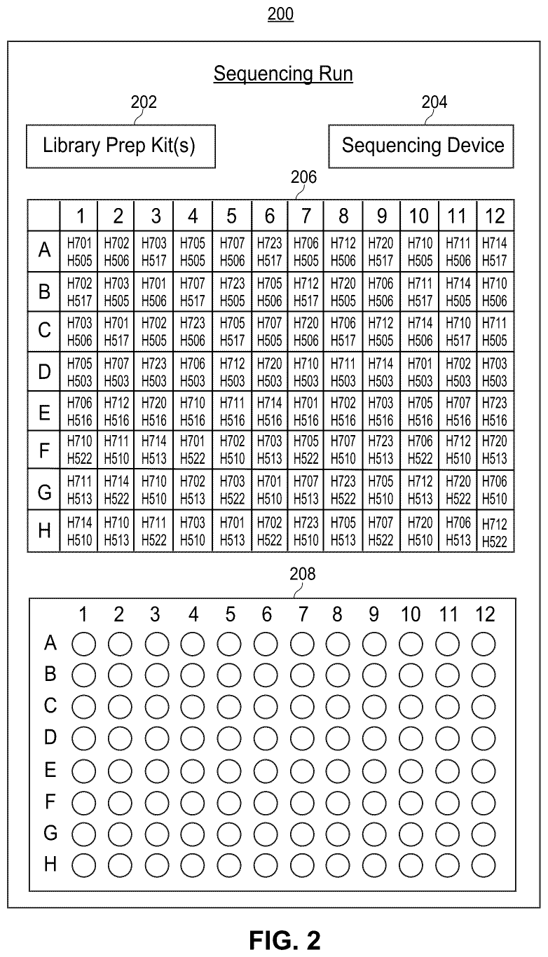

FIG. 2 illustrates an example graphic user interface (GUI) 200 which may be presented on a display screen and/or user interface of a computing device, such as the user computing device 103. For example, the user application 108 may present the GUI. The GUI 200 may include user controls for providing sequencing run data for a sequencing run from a user. For example, the user may enter the library preparation kit type 202 (which includes a list of indices, required reagents, consumables, etc.), the pooling strategy 206, 208 (which includes information regarding the samples in the pool and the arrangement of the pools in for example a 96 well plate), and the sequencing device type 204 via the GUI 200.

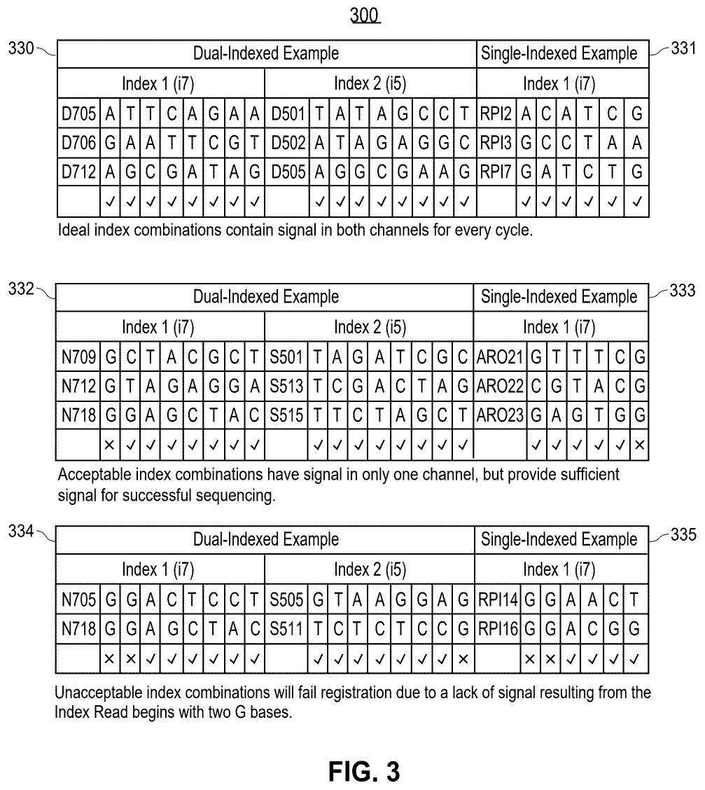

FIG. 3 illustrates example index combinations 300 which may be provided to the user computing device 103 via the user interface. In other implementations, the library preparation system 107 may obtain the index combinations by scanning identification codes (e.g., an RFID tag, a machine-readable barcode, etc.) associated with each index in the index tray to identify the indices. Then the library preparation system 107 may transmit the index combinations to the user computing device 103 or the server device 105, or the controller may analyze the index combinations.

As described herein, indices and combinations of indices are some of several characteristics which must be considered for a successful sequencing run. Depending on e.g., the number of samples, the channel chemistry (e.g., one channel, two channel, four channel) utilized by the sequencing device, and the type of index technique (e.g., combinatorial indexing, unique indexing, single indexing) being utilized, a nucleobase sequence of an index may cause inaccurate sequencing results when used with a nucleobase sequence of another index. Accordingly, for accurate results, indices must be chosen that are compatible with the sequencing run, and compatible with each other. For example, a sequencing run that includes indices that differ by a single base pair or are identical may be inaccurate or unreliable. In another example, a sequencing run in which indices do not have a uniform length will also not be accurate or reliable. In yet another example, a sequencing run having indices that lead to a color imbalance based on the planned sequencer type may be inaccurate or unreliable.

The inherent purpose of an index is to provide users and/or sequencing systems/devices a means to identify nucleobase sequences and accurately demultiplex an unorganized assortment of read data from a sequencing run into organized data. The read data may be organized according to the genetic “tag” (index) attached to a segment of the genetic sample the user is sequencing. Accordingly, the properties of an index (e.g., length, relative length to other indices, sequence, relative sequence to other indices, etc.) are crucial to the compatibility of indices in a pool of samples. For example, identical indices attached to more than one sample may prevent accurate demultiplexing of read data because the read data of two or more samples may be incorrectly lumped into a single data set. Similarly, indices differing by a single nucleobase may prevent accurate demultiplexing of read data because of index or “barcode” collision and the resulting two or more samples being incorrectly lumped into a single data set. Barcode collision occurs because a single mismatch may be allowed in downstream analysis of read data (e.g., demultiplexing) in order to compensate for the possibility of variance/error (e.g., chemical, mechanical) of sequencing devices (e.g., sequencing device 106) during a sequencing run. Additionally, indices of unequal length may prevent accurate demultiplexing of read data for similar reasons.

In some embodiments, the user application 108 obtains genetic sequence data which indicates a nucleobase sequence of each respective index. The user application 108 analyzes the nucleobase sequences of each respective index using several rules to determine whether the nucleobase sequences are compatible with each other. These rules may include: 1) whether two or more nucleobase sequences are identical, 2) whether two or more nucleobase sequences differ by a single nucleobase, 3) whether each nucleobase sequence is a uniform length (e.g., 8 bases long), 4) whether the nucleobase sequences are color balanced at each ordinal position with the nucleobase sequences, 5) whether one or more nucleobase sequences begin with two guanine (“G”) nucleobases in a row, and/or any other suitable rules for determining compatibility.

The user application 108 may apply different rules based on the sequencing run data, such as the library preparation kit type and/or sequencing device type (e.g., “iSeq 100 System”, a “MiniSeq System”), the channel-chemistry type (e.g., one, two, four), the laser type (e.g., color, intensity), the number of dyes required (e.g., 1, 2, 4), dye color(s) (e.g., red and green), the well plate capacity (e.g., 32, 96), the number of bays (e.g., 2, 4), the number of libraries combined in the pool of samples, etc.

For example, if the pool of samples includes three or fewer samples, the user application 108 may determine that the pool has low plexity. Accordingly, the user application 108 may apply a different set of rules for a low plexity sequencing run than a high plexity sequencing run having more than three samples.

In another example, some sequencing devices utilize 2-channel chemistry while others utilize 4-channel chemistry. If the sequencing device type indicates that the sequencing device utilizes 2-channel chemistry, then a “G” nucleobase will not be detected in either blue or green channels. Accordingly, the sequencing device must detect color indicating an adenine (“A”), cytosine (“C”), and/or thymine (“T”) nucleobase at the same ordinal position in another index to detect the absence of color indicating the “G” nucleobase in the index. With 4-channel chemistry this may not be an issue. The user application 108 may not apply this rule if the sequencing device type indicates that the sequencing device utilizes 4-channel chemistry.

As shown in FIG. 3, a sequencing run may utilize dual-indexing (e.g., dual indexing runs 330, 332, 334), in which a sample has an index 1 (17) attached at one end of a sample and an index 2 (15) attached at the other end of the sample, and/or single indexing (e.g., single indexing run 331, 333, 335), in which a sample is attached to an index 1 (17). During a sequencing run, each index is read concurrently so that each ordinal position of the indices is read at the same time (i.e., in the same cycle of a sequencing device cycling through color channels). For example, the single indexing run 331, which shows index “RPI2”, “RPI3”, “RPI7”, may be used in a sequencing run on a sequencing device which may utilize 2-channel chemistry. As shown, the first ordinal position of each index may be, respectively, “G”, “A”, “G”. Because the sequencing device is utilizing 2-channel chemistry, the “A”, corresponding to the nucleobase adenine, signal may be detected in a well for both blue and green channels (i.e., in each image captured during the blue and green channel cycle), and both “G”, corresponding to the nucleobase guanine, signals will not be detected in either blue or green channels (images). It is the absence of a signal which allows the interpretation of a guanine in the first ordinal position of an index. However, for the sequencing device to detect the absence of a signal in a well, it must also detect the signal of an adenine (A), cytosine (C), and/or thymine (T) in another well. This is known as color balancing, and is especially crucial for the first and/or second ordinal positions of an index because once a sequencing run is started, it is calibrated for the remaining cycles of the run. If a sequencing run is not color balanced appropriately, the results of the run may be inaccurate. Accordingly, the single indexing run 331 shows an ideal index combination in which signal is detected in both channels (i.e., the blue or the green), and for every ordinal position.

The user application 108 may obtain these nucleobase sequences corresponding to indices “RPI2”, “RPI3”, “RPI7” as well as other sequencing run data, such as the library preparation kit type, the sequencing device type, the channel-chemistry type (e.g., one, two, four), the laser type (e.g., color, intensity), the number of dyes required (e.g., 1, 2, 4), dye color(s) (e.g., red and green), the well plate capacity (e.g., 32, 96), the number of bays (e.g., 2, 4), etc. Then the user application 108 may analyze the nucleobase sequences for the single indexing run 331 using the set of rules described above. Based on the analysis, the user application 108 may determine that the indices “RPI2”, “RPI3”, “RPI7” are compatible with each other. Accordingly, the user application 108 may present instructions on the user interface to proceed with the sequencing run.

The single indexing run 333 and single indexing run 335, however, show incompatible index combinations. A cycle with signal in only one of the two channels may be an acceptable index combination. For example, the first ordinal position of each index of the single indexing run 333 shows a “G”, “C”, “G”. Accordingly, the first cycle of the corresponding sequence run would only detect signal for the “C” in the image. A cycle with no signal in either channel may be an unacceptable index combination, as shown, for example, in the sixth ordinal position of single indexing run 333 and the first and second ordinal positions of single indexing run 335. Accordingly, single indexing runs 333 and 335 may cause inaccurate sequencing results and the indices (“ARO21”, “ARO22”, “ARO23”) of single indexing run 333 may be incompatible with each other. The indices (“RPI14”, “RPI16”) of single indexing run 335 may also be incompatible with each other.

The user application 108 may obtain the nucleobase sequences corresponding to indices “ARO21”, “ARO22”, “ARO23” as well as other sequencing run data, such as the library preparation kit type, the sequencing device type, the channel-chemistry type (e.g., one, two, four), the laser type (e.g., color, intensity), the number of dyes required (e.g., 1, 2, 4), dye color(s) (e.g., red and green), the well plate capacity (e.g., 32, 96), the number of bays (e.g., 2, 4), etc. Then the user application 108 may analyze the nucleobase sequences for the single indexing run 333 using the set of rules described above. Based on the analysis, the user application 108 may determine that the indices “ARO21”, “ARO22”, “ARO23” are incompatible with each other. Accordingly, the user application 108 may present instructions on the user interface to replace at least one of the plurality of indices with a different index. The user application 108 may also present a recommendation on how to resolve the incompatibility.

To determine how to resolve the incompatibility, the user application 108 may also use the sequencing run data, such as the library preparation kit type, the sequencing device type, the channel-chemistry type (e.g., one, two, four), the laser type (e.g., color, intensity), the number of dyes required (e.g., 1, 2, 4), dye color(s) (e.g., red and green), the well plate capacity (e.g., 32, 96), the number of bays (e.g., 2, 4), etc. The user application 108 may identify the other indices available to the user according to the sequencing run data and may replace a particular index in the sequencing run with one of the other indices. Then the user application 108 may perform the compatibility analysis once again using the different index by applying the set of rules to the adjusted set of indices including the different index.

If the user application 108 determines the adjusted set of indices are compatible with each other, the user application 108 may present a recommendation to replace the particular index with the different index. If the user application 108 determines the adjusted set of indices are incompatible, the user application 108 may replace the particular index with another index available to the user according to the sequencing run data. The user application 108 may also replace a second index in the sequencing run with another index available to the user. The user application 108 may repeat this process until the user application 108 identifies a set of indices which are compatible with each other.

In some implementations, the user application 108 may determine the root cause of the incompatibility (e.g., the indexes at the third ordinal position all have “G” nucleobases) and may search for an index in the available indices which would address the root cause.

In some implementations, the user application 108 may apply one of the rules by determining a ratio of cytosine, adenine, and thymine to guanine for each ordinal position of the nucleobase sequences of the indices in the sequencing run. The ratio may indicate whether the combination of indices are ideal, acceptable, or incompatible, in accordance with the description above. For example, the ratio for the first ordinal position of single indexing run 333 may be 1:2, because there is 1 “C” and 2 “G” nucleobases. The ratio for the sixth ordinal position of single indexing run 333 may be and 0 because there are 0 nucleobases in the sixth ordinal position which are “A,” “C”, or “T.”

The user application 108 may determine, for each ordinal position, whether the ratio exceeds a threshold ratio. Continuing the previous example, the threshold may be zero. Accordingly, any ratio greater than zero may be determined by the user application 108 as ideal and/or acceptable, and any ratio equal to zero as incompatible. In this example, the user application 108 may determine that the sixth ordinal position of single indexing run 333, having a ratio of 0, does not exceed the threshold ratio. In response to determining that the ratio for an ordinal position does not exceed the threshold ratio, the user application 108 may present instructions on the user interface to replace at least one of the indices with a different index.

In some embodiments, the user application 108 may determine a specific index to use to replace one of the indices so that the ratio for each ordinal position exceeds the threshold ratio. In these embodiments, the user application 108 may provide a recommendation to replace one of the indices with the specific index. In various embodiments described herein, the ratio is of cytosine, adenine, and thymine to guanine. However, the ratio may be any method and/or technique for determining whether the combination of indices are ideal, acceptable, or incompatible.

In some embodiments, the user application 108 may apply one of the rules by determining a first ratio of cytosine, adenine, and thymine to guanine for each first ordinal position of the nucleobase sequences of the indices in the sequencing run. The user application 108 may also determine a second ratio of cytosine, adenine, and thymine to guanine for each second ordinal position of the nucleobase sequences of the indices in the sequencing run. The user application 108 may then determine whether the first ratio and the second ratio exceed a threshold ratio. In response to determining the first ratio and the second ratio do not exceed the threshold ratio, the user application 108 may present instructions on the user interface to replace at least one of the indices with a different index. The user application 108 may identify an adjusted set of indices such that at least one of the first ratio or the second ratio exceeds the threshold ratio. Then the user application 108 may present a recommendation on the user interface on how to resolve the incompatibility by using the adjusted set of indices.

For example, if at least one index in the sequencing run in the first or second ordinal positions includes a “C,” “T,” or “A,” the user application 108 may determine that the indices are compatible provided that the indices are compliant with the other rules. However, if all of the nucleobases in the first and second ordinal positions are “G,” the user application 108 may determine that the indices are incompatible.

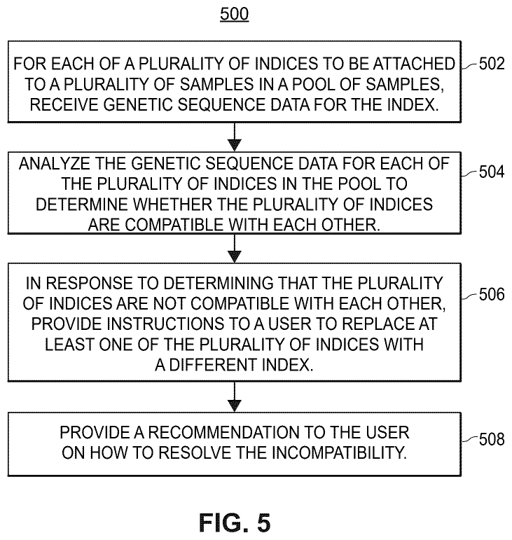

FIG. 5 is a flow diagram of an example method 500 for determining the validity of indexes attached to a pool of samples. One or more steps of the computer-implemented method of 500 may be implemented as a set of instructions stored on a computer-readable memory and executable on one or more processors. The computer-implemented method 500 may operate in the environment illustrated in FIG. 1. For example, the method 500 may be implemented by the user computing device 103 of FIG. 1. In another example, the method 500 may be implemented by the server 105. In yet another example, the method 500 may be implemented by the library preparation system 107. More generally, the method 500 may be implemented by any suitable combination of the user computing device 103, the server 105, and/or the library preparation system.

At block 502, the user computing device 103 receives genetic sequence data for each of a plurality of indices to be attached to samples in a pool of samples. For example, the genetic sequence data may include an identifier of each of the indices in the sequencing run, the nucleobase sequence for each index, etc. The user computing device 103 may also receive additional sequencing run data for the pool of samples, such as the library preparation kit type, the sequencing device type, the channel-chemistry type, the laser type, the number of dyes required, the dye color(s), the well plate capacity, the number of bays, the number of libraries combined in the pool of samples, etc.

Then at block 504, the user computing device 103 analyzes the genetic sequence data for each of the indices in the pool to determine whether the indices are compatible with each other. For example, the user computing device 103 may apply a set of rules to the nucleobase sequences for the indices to determine compatibility. The user computing device 103 may apply different rules based on the sequencing run data. For example, the user computing device 103 may store several sets of rules and may selects one of the sets of rules to use according to the sequencing run data.

An example set of rules may include 1) whether two or more nucleobase sequences are identical, 2) whether two or more nucleobase sequences differ by a single nucleobase, 3) whether each nucleobase sequence is a uniform length (e.g., 8 bases long), 4) whether the nucleobase sequences are color balanced at each ordinal position with the nucleobase sequences, and/or 5) whether one or more nucleobase sequences begin with two guanine (“G”) nucleobases in a row. The user computing device 103 may apply each of the rules in the set to determine compatibility. If at least one of the rules is violated, the user computing device 103 may determine that the indices are incompatible with each other.

In response to determining that the indices are incompatible with each other, the user computing device 103 may provide instructions to the user to replace at least one of the indices with a different index (block 506). For example, the user computing device 103 may present the instructions on a user interface.

The user computing device 103 may also determine how to resolve the incompatibility. For example, the user computing device 103 may use the sequencing run data to identify the other indices available to the user. The user computing device 103 may replace a particular index in the sequencing run with one of the other indices. Then the user application 108 may perform the compatibility analysis once again using the different index by applying the set of rules to the adjusted set of indices including the different index. In some implementations, the user computing device 103 may determine the root cause of the incompatibility (e.g., the indexes at the third ordinal position all have “G” nucleobases) and may search for an index in the available indices which would address the root cause.

In any event, the user computing device 103 may provide a recommendation to the user on how to resolve the incompatibility (block 508). For example, the user computing device 103 may present the recommendation on the user interface. The recommendation may include the index(es) to remove from the sequencing run and the other index(es) available to the user to include in the sequencing run to form an adjusted set of indices.

Example 1. A method for determining the validity of indexes attached to a pool of samples, the method comprising: for each of a plurality of indices to be attached to a plurality of samples in a pool of samples, receiving, at one or more processors, genetic sequence data for the index; analyzing, by the one or more processors, the genetic sequence data for each of the plurality of indices in the pool to determine whether the plurality of indices are compatible with each other; and in response to determining that the plurality of indices are not compatible with each other, providing, by the one or more processors, instructions to a user to replace at least one of the plurality of indices with a different index.

Example 2. The method of example 1, further comprising: providing, by the one or more processors, a recommendation to the user on how to resolve the incompatibility.

Example 3. The method of example 1 or example 2, wherein the one or more processors are included in a library preparation system, wherein the at least one index is replaced with the different index, and further comprising: attaching, by the library preparation system, the different index to one of the plurality of samples in the pool.

Example 4. The method of any one of the preceding examples, wherein the genetic sequence data indicates a nucleobase sequence of each respective index, and further comprising: determining, by the one or more processors, whether two or more nucleobase sequences are identical; and in response to determining that two or more nucleobase sequences are identical, providing, by the one or more processors, instructions to the user to replace at least one of the two or more nucleobase sequences.

Example 5. The method of any one of the preceding examples, wherein the genetic sequence data indicates a nucleobase sequence of each respective index, and further comprising: determining, by the one or more processors, whether two or more nucleobase sequences differ by a single nucleobase; and in response to determining that two or more nucleobase sequences differ by a single nucleobase, providing, by the one or more processors, instructions to the user to replace at least one of the two or more nucleobase sequences.

Example 6. The method of any one of the preceding examples, wherein the genetic sequence data indicates a nucleobase sequence of each respective index, and further comprising: determining, by the one or more processors, whether each nucleobase sequence is a uniform length; and in response to determining that the length of each nucleobase sequence is not uniform, providing, by the one or more processors, instructions to the user to replace at least one of the plurality of indices with a different index so that the length of each nucleobase sequence is uniform.

Example 7. The method of any one of the preceding examples, wherein the genetic sequence data indicates a nucleobase sequence of each respective index, and further comprising: for each ordinal position of the nucleobase sequences of the plurality of indices, determining, by the one or more processors, a ratio of cytosine, adenine, and thymine to guanine; for each ordinal position, determining, by the one or more processors, whether the ratio exceeds a threshold ratio; and in response to determining that the ratio for one or more ordinal positions does not exceed the threshold ratio, providing, by the one or more processors, instructions to the user to replace at least one of the plurality of indices with a different index so that the ratio for each ordinal position exceeds the threshold ratio.

Example 8. The method of any one of the preceding examples, wherein the genetic sequence data indicates a nucleobase sequence of each respective index, wherein each nucleobase sequence comprises a first ordinal position and a second ordinal position, and further comprising: for each first ordinal position, determining, by the one or more processors, a first ratio of cytosine, adenine, and thymine to guanine; for each second ordinal position, determining, by the one or more processors, a second ratio of cytosine, adenine, and thymine to guanine; determining, by the one or more processors, whether the first ratio and the second ratio exceed a threshold ratio; and in response to determining the first ratio and the second ratio do not exceed the threshold ratio, providing, by the one or more processors, instructions to the user to replace at least one of the plurality of indices with a different index so that at least one of the first ratio or the second ratio exceeds the threshold ratio.

Example 9. The method of any one of the preceding examples, further comprising: determining, by the one or more processors, whether the plurality of indices are attached to three or fewer samples in the pool of samples; in response to determining the plurality of indices are attached to three or fewer samples in the pool of samples, analyzing, by the one or more processors, the genetic sequence data for each of the plurality of indices in the pool to determine whether the plurality of indices are compatible with each other based on the plurality of indices being attached to three or fewer samples; and in response to determining that the plurality of indices are not compatible with each other based on the plurality of indices being attached to three or fewer samples, providing, by the one or more processors, instructions to the user to replace at least one of the plurality of indices with a different index.

Example 10. The method of any one of the preceding examples, wherein a sequencer system is configured to sequence the plurality of samples in a pool of samples, and further comprising: receiving, at one or more processors, sequencer data for the sequencer system; analyzing, by the one or more processors, the sequencer data and the genetic sequence data for each of the plurality of indices in the pool to determine whether the plurality of indices are compatible with the sequencer system; in response to determining that the plurality of indices are not compatible with the sequencer system, providing, by the one or more processors, instructions to the user to replace at least one of the plurality of indices with a different index.

Example 11. The method of any one of the preceding examples, wherein each of the plurality of indices is associated with an identification code, and further comprising: obtaining, by the one or more processors, the identification code of one or more of the plurality of indices; and determining, by the one or more processors, a nucleobase sequence corresponding to the identification code.

Example 12. A system for determining the validity of indexes attached to a pool of samples, the system comprising: one or more processors; and a non-transitory computer-readable memory storing instructions thereon that, when executed by the one or more processors, cause the system to: for each of a plurality of indices to be attached to a plurality of samples in a pool of samples, receive genetic sequence data for the index, analyze the genetic sequence data for each of the plurality of indices in the pool to determine whether the plurality of indices are compatible with each other, and in response to determining that the plurality of indices are not compatible with each other, provide, via a user interface, instructions to a user to replace at least one of the plurality of indices with a different index.

Example 13. The system of example 12, wherein the instructions further cause system to: provide, via the user interface, a recommendation to the user on how to resolve the incompatibility.

Example 14. The system of example 12 or example 13, wherein the at least one index is replaced with the different index, and wherein the instructions further cause the system to: cause a library preparation system communicatively coupled to the one or more processors to replace the at least one index with the different index by attaching the different index to one of the plurality of samples in the pool.

Example 15. The system of any one of examples 12 to 14, wherein the genetic sequence data indicates a nucleobase sequence of each respective index, and wherein the instructions further cause the system to: determine whether two or more nucleobase sequences are identical; and in response to determining that two or more nucleobases are identical, provide, via the user interface, instructions to the user to replace at least one of the two or more nucleobase sequences.

Example 16. The system of any one of examples 12 to 15, wherein the genetic sequence data indicates a nucleobase sequence of each respective index, and wherein the instructions further cause the system to: determine whether two or more nucleobase sequences differ by a single nucleobase; and in response to determining that two or more nucleobase sequences differ by a single nucleobase, provide, via the user interface, instructions to the user to replace at least one of the two or more nucleobase sequences.

Example 17. The system of any one of examples 12 to 16, wherein the genetic sequence data indicates a nucleobase sequence of each respective index, and wherein the instructions further cause the system to: determine whether each nucleobase sequence is a uniform length, and in response to determining that the length of each nucleobase sequence is not uniform, provide instructions to the user to replace at least one of the plurality of indices with a different index so that the length of each nucleobase sequence is uniform.

Example 18. The system of any one of examples 12 to 17, wherein the genetic sequence data indicates a nucleobase sequence of each respective index, and wherein the instructions further cause the system to: determine, for each ordinal position of the nucleobase sequences of the plurality of indices, a ratio of cytosine, adenine, and thymine to guanine; determine, for each ordinal position, whether the ratio exceeds a threshold ratio; and in response to determining that the ratio for one or more ordinal positions does not exceed the threshold ratio, provide, via the user interface, instructions to the user to replace at least one of the plurality of indices with a different index so that the ratio for each ordinal position exceeds the threshold ratio.

Example 19. The system of any one of examples 12 to 18, wherein the genetic sequence data indicates a nucleobase sequence of each respective index, wherein each nucleobase sequence comprises a first ordinal position and a second ordinal position, and wherein the instructions further cause the system to: determine, for each first ordinal position, a first ratio of cytosine, adenine, and thymine to guanine; determine, for each second ordinal position, a second ratio of cytosine, adenine, and thymine to guanine; determine whether the first ratio and the second ratio exceed a threshold ratio; and in response to determining the first ratio and the second ratio do not exceed the threshold ratio, provide instructions to the user to replace at least one of the plurality of indices with a different index so that at least one of the first ratio or the second ratio exceeds the threshold ratio.

Example 20. A non-transitory computer-readable memory storing instructions for determining the validity of indexes attached to a pool of samples, that when executed by one or more processors, cause the one or more processors to: for each of a plurality of indices to be attached to a plurality of samples in a pool of samples, receive genetic sequence data for the index; analyze the genetic sequence data for each of the plurality of indices in the pool to determine whether the plurality of indices are compatible with each other; and in response to determining that the plurality of indices are not compatible with each other, provide instructions to a user to replace at least one of the plurality of indices with a different index.

Although the disclosure herein sets forth a detailed description of numerous different embodiments, it should be understood that the legal scope of the description is defined by the words of the claims set forth at the end of this patent and equivalents. The detailed description is to be construed as exemplary only and does not describe every possible embodiment since describing every possible embodiment would be impractical. Numerous alternative embodiments may be implemented, using either current technology or technology developed after the filing date of this patent, which would still fall within the scope of the claims.

The following additional considerations apply to the foregoing discussion. Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules may provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and may operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location, while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other embodiments, the one or more processors or processor-implemented modules may be distributed across a number of geographic locations.

This detailed description is to be construed as exemplary only and does not describe every possible embodiment, as describing every possible embodiment would be impractical, if not impossible. A person of ordinary skill in the art may implement numerous alternate embodiments, using either current technology or technology developed after the filing date of this application.

Those of ordinary skill in the art will recognize that a wide variety of modifications, alterations, and combinations can be made with respect to the above described embodiments without departing from the scope of the invention, and that such modifications, alterations, and combinations are to be viewed as being within the ambit of the inventive concept.

The patent claims at the end of this patent application are not intended to be construed under 35 U.S.C. § 112 (f) unless traditional means-plus-function language is expressly recited, such as “means for” or “step for” language being explicitly recited in the claim(s). The systems and methods described herein are directed to an improvement to computer functionality, and improve the functioning of conventional computers.

What is claimed is:

1. A method for determining the validity of indexes attached to a pool of samples, the method comprising:
for each of a plurality of indices to be attached to a plurality of samples in a pool of samples, receiving, at one or more processors, genetic sequence data for the index;
analyzing, by the one or more processors, the genetic sequence data for each of the plurality of indices in the pool to determine whether the plurality of indices are compatible with each other; and
in response to determining that the plurality of indices are not compatible with each other, providing, by the one or more processors, instructions to a user to replace at least one of the plurality of indices with a different index.

2. The method of claim 1, further comprising:
providing, by the one or more processors, a recommendation to the user on how to resolve the incompatibility.

3. The method of claim 1, wherein the one or more processors are included in a library preparation system, wherein the at least one index is replaced with the different index, and further comprising:
attaching, by the library preparation system, the different index to one of the plurality of samples in the pool.

4. The method of claim 1, wherein the genetic sequence data indicates a nucleobase sequence of each respective index, and further comprising:
determining, by the one or more processors, whether two or more nucleobase sequences are identical; and
in response to determining that two or more nucleobase sequences are identical, providing, by the one or more processors, instructions to the user to replace at least one of the two or more nucleobase sequences.

5. The method of claim 1, wherein the genetic sequence data indicates a nucleobase sequence of each respective index, and further comprising:
determining, by the one or more processors, whether two or more nucleobase sequences differ by a single nucleobase; and
in response to determining that two or more nucleobase sequences differ by a single nucleobase, providing, by the one or more processors, instructions to the user to replace at least one of the two or more nucleobase sequences.

6. The method of claim 1, wherein the genetic sequence data indicates a nucleobase sequence of each respective index, and further comprising:
determining, by the one or more processors, whether each nucleobase sequence is a uniform length; and
in response to determining that the length of each nucleobase sequence is not uniform, providing, by the one or more processors, instructions to the user to replace at least one of the plurality of indices with a different index so that the length of each nucleobase sequence is uniform.

7. The method of claim 1, wherein the genetic sequence data indicates a nucleobase sequence of each respective index, and further comprising:
for each ordinal position of the nucleobase sequences of the plurality of indices, determining, by the one or more processors, a ratio of cytosine, adenine, and thymine to guanine;
for each ordinal position, determining, by the one or more processors, whether the ratio exceeds a threshold ratio; and
in response to determining that the ratio for one or more ordinal positions does not exceed the threshold ratio, providing, by the one or more processors, instructions to the user to replace at least one of the plurality of indices with a different index so that the ratio for each ordinal position exceeds the threshold ratio.

8. The method of claim 1, wherein the genetic sequence data indicates a nucleobase sequence of each respective index, wherein each nucleobase sequence comprises a first ordinal position and a second ordinal position, and further comprising:
for each first ordinal position, determining, by the one or more processors, a first ratio of cytosine, adenine, and thymine to guanine;
for each second ordinal position, determining, by the one or more processors, a second ratio of cytosine, adenine, and thymine to guanine;
determining, by the one or more processors, whether the first ratio and the second ratio exceed a threshold ratio; and
in response to determining the first ratio and the second ratio do not exceed the threshold ratio, providing, by the one or more processors, instructions to the user to replace at least one of the plurality of indices with a different index so that at least one of the first ratio or the second ratio exceeds the threshold ratio.

9. The method of claim 1, further comprising:
determining, by the one or more processors, whether the plurality of indices are attached to three or fewer samples in the pool of samples;
in response to determining the plurality of indices are attached to three or fewer samples in the pool of samples, analyzing, by the one or more processors, the genetic sequence data for each of the plurality of indices in the pool to determine whether the plurality of indices are compatible with each other based on the plurality of indices being attached to three or fewer samples; and
in response to determining that the plurality of indices are not compatible with each other based on the plurality of indices being attached to three or fewer samples, providing, by the one or more processors, instructions to the user to replace at least one of the plurality of indices with a different index.

10. The method of claim 1, wherein a sequencer system is configured to sequence the plurality of samples in a pool of samples, and further comprising:
receiving, at one or more processors, sequencer data for the sequencer system;
analyzing, by the one or more processors, the sequencer data and the genetic sequence data for each of the plurality of indices in the pool to determine whether the plurality of indices are compatible with the sequencer system;
in response to determining that the plurality of indices are not compatible with the sequencer system, providing, by the one or more processors, instructions to the user to replace at least one of the plurality of indices with a different index.

11. The method of claim 1, wherein each of the plurality of indices is associated with an identification code, and further comprising:
obtaining, by the one or more processors, the identification code of one or more of the plurality of indices; and
determining, by the one or more processors, a nucleobase sequence corresponding to the identification code.

12. A system for determining the validity of indexes attached to a pool of samples, the system comprising:
one or more processors; and
a non-transitory computer-readable memory storing instructions thereon that, when executed by the one or more processors, cause the system to:
for each of a plurality of indices to be attached to a plurality of samples in a pool of samples, receive genetic sequence data for the index,
analyze the genetic sequence data for each of the plurality of indices in the pool to determine whether the plurality of indices are compatible with each other, and
in response to determining that the plurality of indices are not compatible with each other, provide, via a user interface, instructions to a user to replace at least one of the plurality of indices with a different index.

13. The system of claim 12, wherein the instructions further cause system to:
provide, via the user interface, a recommendation to the user on how to resolve the incompatibility.

14. The system of claim 12, wherein the at least one index is replaced with the different index, and wherein the instructions further cause the system to:
cause a library preparation system communicatively coupled to the one or more processors to replace the at least one index with the different index by attaching the different index to one of the plurality of samples in the pool.

15. The system of claim 12, wherein the genetic sequence data indicates a nucleobase sequence of each respective index, and wherein the instructions further cause the system to:
determine whether two or more nucleobase sequences are identical; and
in response to determining that two or more nucleobases are identical, provide, via the user interface, instructions to the user to replace at least one of the two or more nucleobase sequences.

16. The system of claim 12, wherein the genetic sequence data indicates a nucleobase sequence of each respective index, and wherein the instructions further cause the system to:
determine whether two or more nucleobase sequences differ by a single nucleobase; and
in response to determining that two or more nucleobase sequences differ by a single nucleobase, provide, via the user interface, instructions to the user to replace at least one of the two or more nucleobase sequences.

17. The system of claim 12, wherein the genetic sequence data indicates a nucleobase sequence of each respective index, and wherein the instructions further cause the system to:
determine whether each nucleobase sequence is a uniform length, and
in response to determining that the length of each nucleobase sequence is not uniform, provide instructions to the user to replace at least one of the plurality of indices with a different index so that the length of each nucleobase sequence is uniform.

18. The system of claim 12, wherein the genetic sequence data indicates a nucleobase sequence of each respective index, and wherein the instructions further cause the system to:
determine, for each ordinal position of the nucleobase sequences of the plurality of indices, a ratio of cytosine, adenine, and thymine to guanine;
determine, for each ordinal position, whether the ratio exceeds a threshold ratio; and
in response to determining that the ratio for one or more ordinal positions does not exceed the threshold ratio, provide, via the user interface, instructions to the user to replace at least one of the plurality of indices with a different index so that the ratio for each ordinal position exceeds the threshold ratio.

19. The system of claim 12, wherein the genetic sequence data indicates a nucleobase sequence of each respective index, wherein each nucleobase sequence comprises a first ordinal position and a second ordinal position, and wherein the instructions further cause the system to:
determine, for each first ordinal position, a first ratio of cytosine, adenine, and thymine to guanine;
determine, for each second ordinal position, a second ratio of cytosine, adenine, and thymine to guanine;
determine whether the first ratio and the second ratio exceed a threshold ratio; and
in response to determining the first ratio and the second ratio do not exceed the threshold ratio, provide instructions to the user to replace at least one of the plurality of indices with a different index so that at least one of the first ratio or the second ratio exceeds the threshold ratio.

20. A non-transitory computer-readable memory storing instructions for determining the validity of indexes attached to a pool of samples, that when executed by one or more processors, cause the one or more processors to:
for each of a plurality of indices to be attached to a plurality of samples in a pool of samples, receive genetic sequence data for the index;
analyze the genetic sequence data for each of the plurality of indices in the pool to determine whether the plurality of indices are compatible with each other; and
in response to determining that the plurality of indices are not compatible with each other, provide instructions to a user to replace at least one of the plurality of indices with a different index.
